(19) United States
(12) Patent Application Publication
(10) Pub. No.: US 2024/0208980 A1
(43) Pub. Date: Jun. 27, 2024

(54) Fostemsavir Intermediates and Process for the Preparation Thereof

(71) Applicant: Navinta, LLC, Ewing, NJ (US)

(72) Inventors: Pankaj Vasudev Parmar, Vadodara (IN); John Muthiah Raja Jeyakumar, Vadodara (IN); Subba Narasimhulu Porala, Hyderabad (IN); Mukesh Parshottambhai Suvagia, Vadodara (IN); Piyushkumar Parshotam Sangani, Vadodara (IN)

(21) Appl. No.: 18/542,121

(22) Filed: Dec. 15, 2023

Related U.S. Application Data
(60) Provisional application No. 63/433,724, filed on Dec. 19, 2022.

(51) Int. Cl. C07D 487/04

(52) U.S. Cl. CPC C07D 487/04

(57) ABSTRACT

The invention relates to novel Fostemsavir intermediates and process for the preparation of novel Fostemsavir intermediates. The invention also relates to the use of novel Fostemsavir intermediates in the manufacturing of highly pure Fostemsavir or pharmaceutically acceptable salts thereof.

### FIELD OF THE INVENTION

The present invention relates to novel Fostemsavir intermediates and the process for the preparation thereof. The present invention also relates to the use of novel Fostemsavir intermediates in the manufacturing of highly pure Fostemsavir or pharmaceutically acceptable salts thereof.

### BACKGROUND OF THE INVENTION

Fostemsavir Tromethamine (Brand Name: RUKOBIA®) from VIIV HEALTHCARE was first approved by USFDA in 2020 for the treatment of HIV-1 infection in heavily treatment-experienced adults with multidrug-resistant HIV-1 infections. It is a first-in-class HIV attachment inhibitor. The chemical name of Fostemsavir Tromethamine is (3-((4-benzoyl-1-piperazinyl)(oxo)acetyl)-4-methoxy-7-(3-methyl-1H-1,2,4-triazol-1-yl)-1H-pyrrolo[2,3-c]-pyridin-1-yl)methyl dihydrogen phosphate, 2-amino-2-(hydroxymethyl)-1,3-propanediol. Fostemsavir in Adults with Multidrug-Resistant HIV-1 Infection, Michael Kozal et. al., The New England Journal of Medicine, 382; 13, Mar. 26, 2020, 1232-1243.

Fostemsavir is a prodrug of Temsavir (BMS-626529), which is chemically known as 1-(4-benzoylpiperazin-1-yl)-2-[4-methoxy-7-(3-methyl-1,2,4-triazol-1-yl)-1H-pyrrolo[2,3-c]pyridin-3-yl]ethane-1,2-dione.

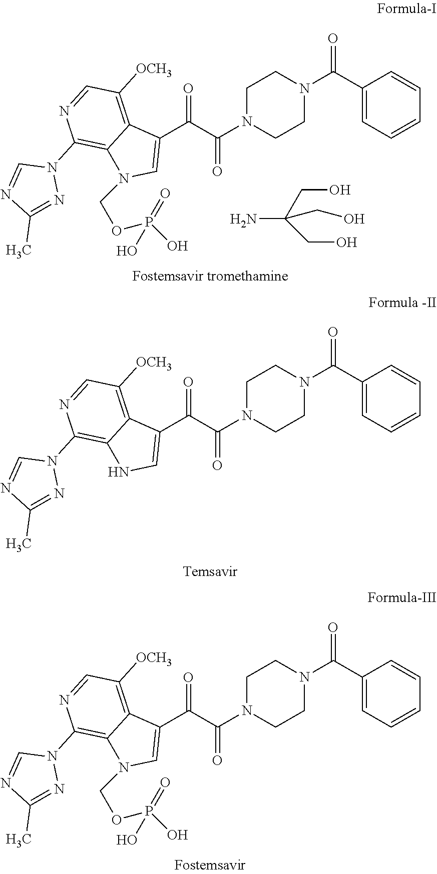 [SMILES: COc1cnc(-n2cnc(C)n2)c2c1c(C(=O)C(=O)N1CCN(C(=O)c3ccccc3)CC1)cn2COP(=O)(O)O ; NC(CO)(CO)CO ; COc1cnc(-n2cnc(C)n2)c2[nH]cc(C(=O)C(=O)N3CCN(C(=O)c4ccccc4)CC3)c12 ; COc1cnc(-n2cnc(C)n2)c2c1c(C(=O)C(=O)N1CCN(C(=O)c3ccccc3)CC1)cn2COP(=O)(O)O]

U.S. Pat. No. 7,745,625 discloses the Fostemsavir Tromethamine and the process for the preparation of Fostemsavir Tromethamine represented below (Scheme-1):

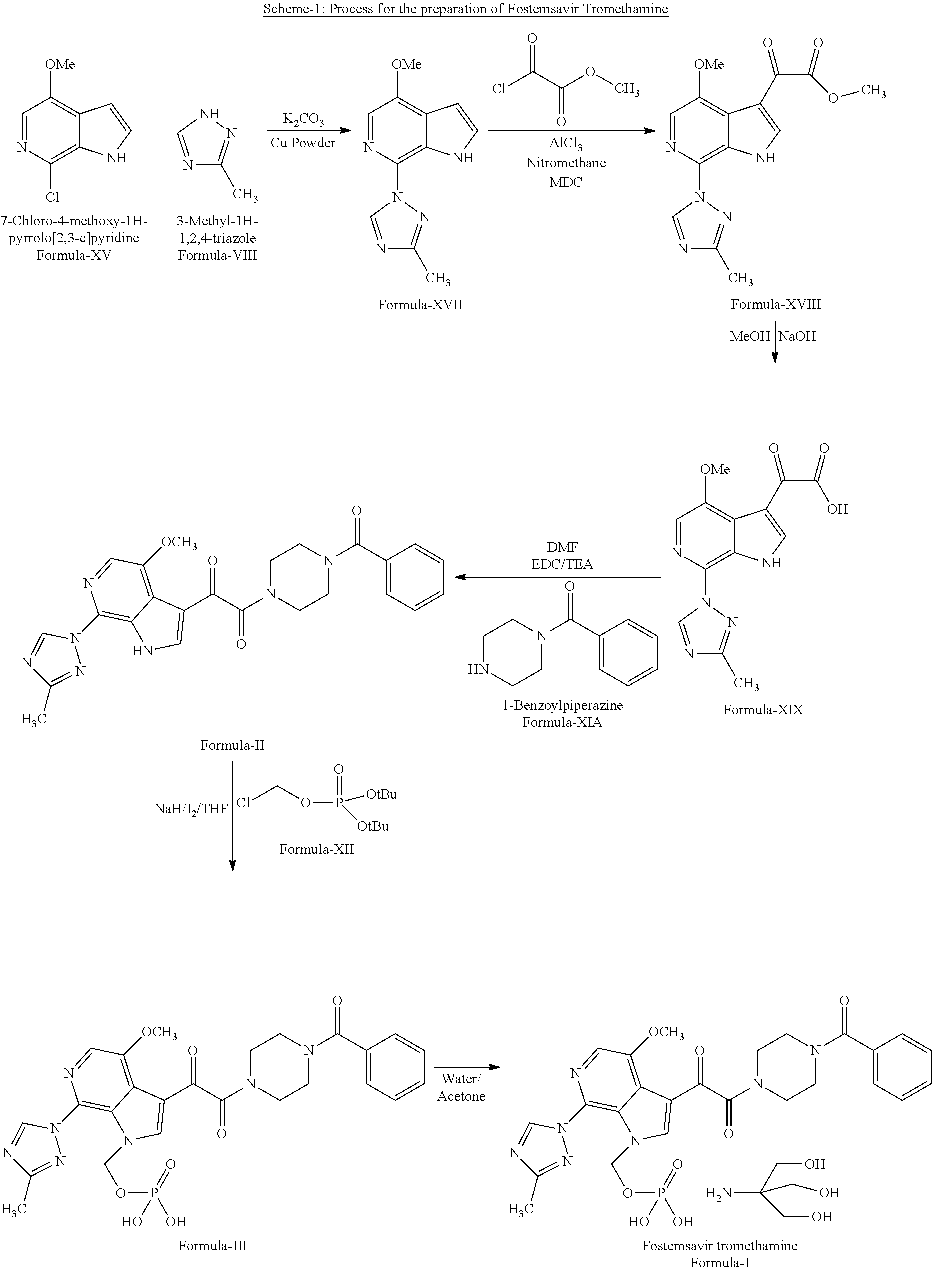

Fostemsavir tromethamine prepared by Scheme-1 has the following drawbacks:
a) During the process, there is formation of about 40% undesired positional isomer of triazole at stage 1; and
b) Multiple purifications are required at subsequent stages to reduce the presence of this undesired isomer resulting in significant yield loss.

U.S. Pat. No. 7,745,625 and Eastgate et. al. (Organic Process Research & Development, 2017, Vol. 21) disclose another process for the preparation of Fostemsavir Tromethamine by introducing a triazole moiety at a later stage after the rest of the skeleton was assembled as depicted in the below Scheme-2.

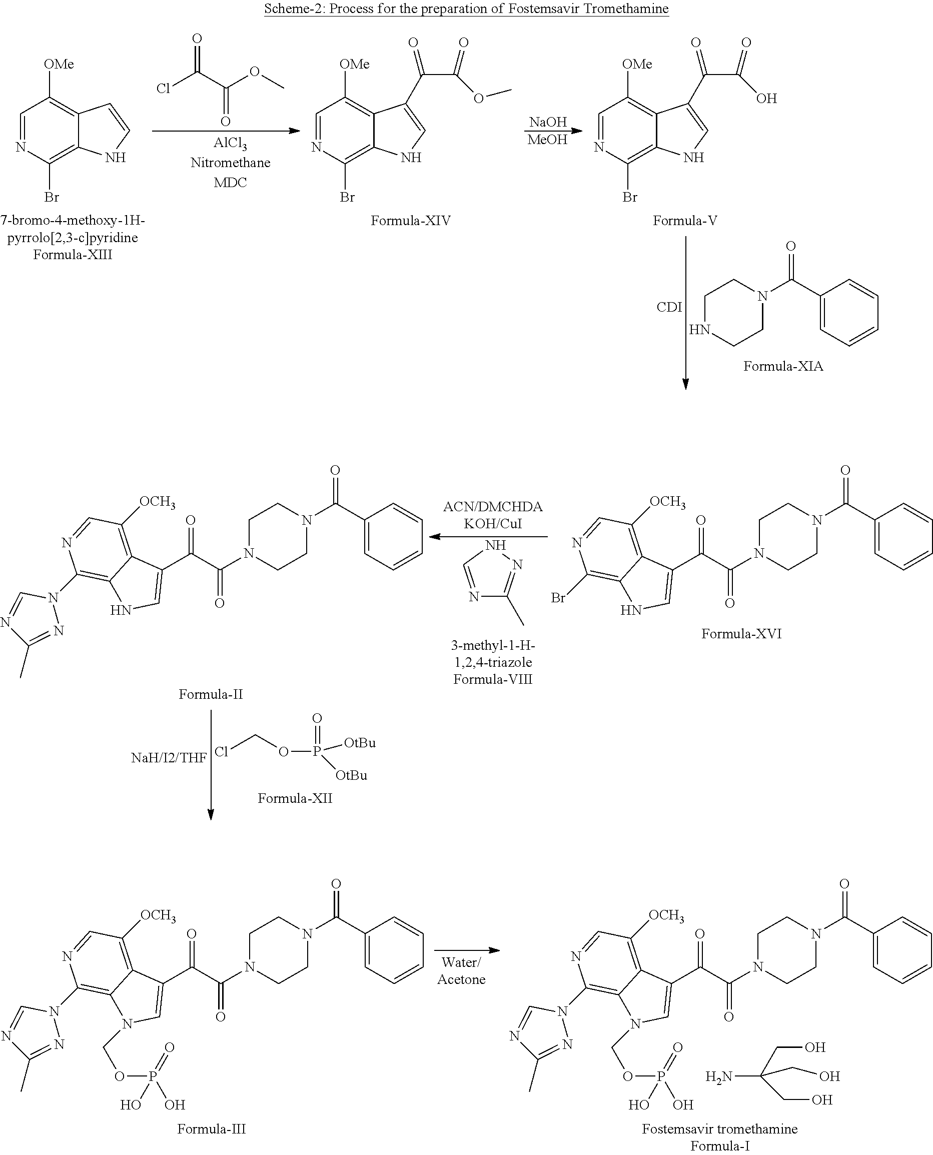

Fostemsavir tromethamine prepared by scheme-2 has the following drawbacks—although the process disclosed in Scheme-2 is better than Scheme-1, it still gives about 6% of undesired triazole isomer in stage 4, which must be removed by purification.

There are several prior arts references that disclose the use of a different protecting group, other than Cbz, for the preparation of Fostemsavir tromethamine. For example, PCT publication WO2004/011425 in Scheme-1 discloses use of Boc-protected piperazine compound with di-keto compound, which further undergoes deprotection of Boc group to obtain the desired compound. The Boc-protected process has disadvantages such as it results in formation of uncontrolled undesired isomers during the process.

Overall, the processes reported in the prior art have the following drawbacks:
All of the reported processes are not efficient to produce a highly pure Fostemsavir tromethamine;
The reported processes lack control over the impurity formation and its purification;
The reported processes are multistep processes that require costly intermediates and are time consuming;
The reported processes result in formation of undesired isomers; and
The reported processes require multiple purifications at different stages, which result in yield loss of final compound.

Thus, there remains a need to provide a robust process for the preparation of novel Fostemsavir intermediates, which can be easily used in the preparation of Fostemsavir or pharmaceutically acceptable salts.

### SUMMARY OF THE INVENTION

A main objective of the present invention is to provide a novel Fostemsavir intermediate represented by Formula-VIIA:

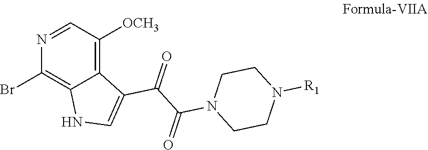

wherein R¹=

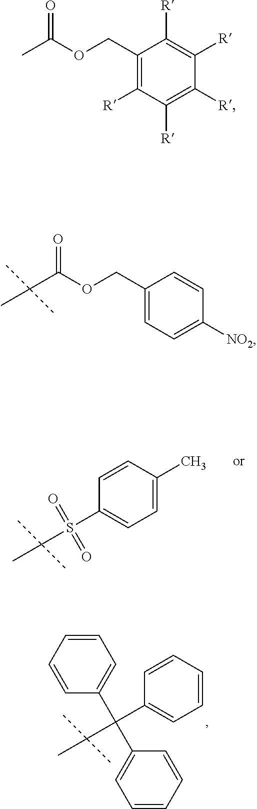

and
wherein
R′=H, F, Cl, Br, I, C₁-C₆ alkyl, Aryl, —NO₂, —OR″, —COOR″ or —NR″₂, and R″=H, C₁-C₆ alkyl or Aryl.

Another objective of the present invention is to provide a novel Fostemsavir intermediate represented by Formula-IXA

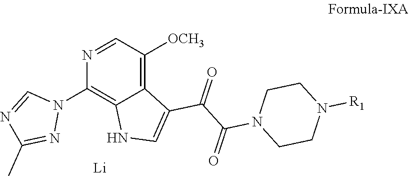

wherein R¹=

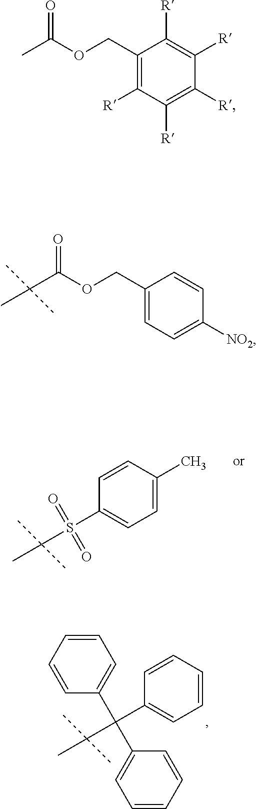

and
wherein
R′=H, F, Cl, Br, I, C₁-C₆ alkyl, Aryl, —NO₂, —OR″, —COOR″ or —NR″₂, and
R″=H, C₁-C₆ alkyl or Aryl.

Yet another objective of the present invention is to provide a process for preparation of 1-(4-methoxy-7-(3-methyl-1H-1,2,4-triazol-1-yl)-1H-pyrrolo[2,3-c]pyridin-3-yl)-2-(piperazin-1-yl)ethane-1,2-dione of Formula-X including the steps of:
(a) reacting 2-(7-bromo-4-methoxy-1H-pyrrolo[2,3-c]pyridin-3-yl)-2-oxoacetic acid of Formula-V with 1-Cbz piperazine of Formula-VI in presence of coupling agent, base and solvent to obtain 1-(7-bromo-4-methoxy-1H-pyrrolo[2,3-c]pyridin-3-yl)-2-(4-substituted-piperazin-1-yl)ethane-1,2-dione of Formula-VIIA;
(b) reacting compound of Formula-VIIA as obtained in step (a) with 3-Methyl-1H-1,2,4-triazole of Formula-VIII in presence of DMCHDA, Solvent, base and metal catalyst to obtain 1-(4-substituted-piperazin-1-yl)-2-(4-methoxy-7-(3-methyl-1H-1,2,4-triazol-1-yl)-1H-pyrrolo[2,3-c]pyridin-3-yl)ethane-1,2-dione lithium salt of Formula-IXA; and
(c) reducing compound of Formula-IXA in presence of a reagent and a solvent to obtain 1-(4-methoxy-7-(3-methyl-1H-1,2,4-triazol-1-yl)-1H-pyrrolo[2,3-c]pyridin-3-yl)-2-(piperazin-1-yl)ethane-1,2-dione of Formula-X.

Further objective of the present invention is to provide a process for preparation of Fostemsavir or pharmaceutically acceptable salts thereof from intermediate compound of formula-X as obtained in above mentioned step-c, which includes the steps of:
(d) benzoylating compound of Formula-X as obtained in step-c with a coupling agent, base and solvent to obtain Temsavir of Formula-Il;
(e) treating Temsavir of Formula-Il as obtained in step-d with di-tert-butyl (chloromethyl) phosphate in presence of acetonitrile, Potassium carbonate and Tetraethylammonium iodide to obtain in situ compound of Formula-IV;
(f) adding water and acetone to in-situ compound of Formula-IV To obtain in-situ Fostemsavir of Formula-III; and
(g) converting in-situ Fostemsavir to Fostemsavir tromethamine of Formula-I in presence of Tromethamine.

Yet another objective of the present invention is to provide a process for the preparation of Fostemsavir or pharmaceutically acceptable salt by using a novel Fostemsavir intermediate represented by Formula-VII and Formula-IX.

A further objective of the present invention is to provide an economical, efficient and viable process for the preparation of Fostemsavir intermediate represented by Formula-X.

Yet another objective of the present invention is to provide Fostemsavir intermediate of Formula-VII prepared by the process of the present invention having HPLC purity of greater than or equal to 99.58%.

An additional objective of the present invention is to provide Fostemsavir intermediate of Formula-IX prepared by the process of the present invention having HPLC purity of greater than or equal to 99.56%.

### BRIEF DESCRIPTION OF THE DRAWINGS

FIG. 1 shows HPLC pattern of Fostemsavir intermediate of Formula-VII.

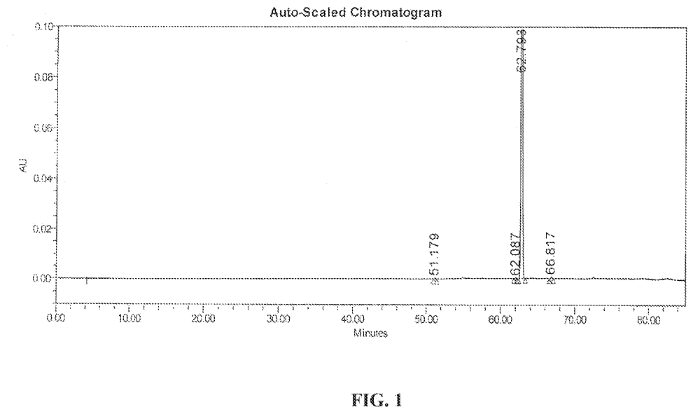

FIG. 2 shows HPLC pattern of Fostemsavir intermediate of Formula-IX.

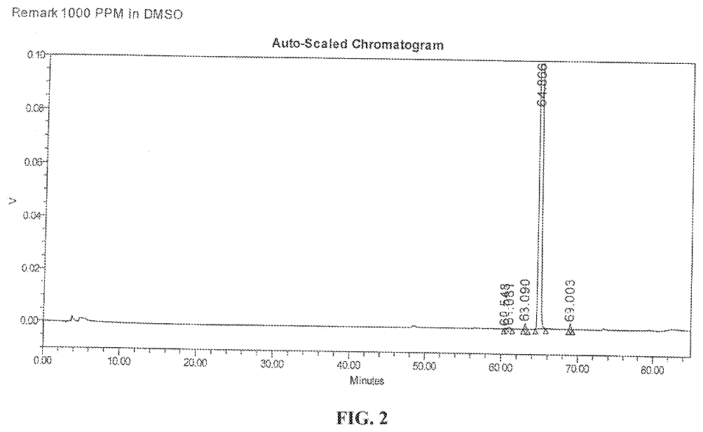

### DETAILED DESCRIPTION OF THE INVENTION

The terms “comprising” and “comprises” refer to the elements recited, or their equivalents in structure or function, plus any other element or elements which are not recited.

The terms “having” and “including” are to be construed as open ended. All ranges recited herein include the endpoints, including those that recite a range between two values. Whether so indicated or not, all values recited herein are approximate as defined by the circumstances, including the degree of expected experimental error, technique error, and instrument error for a given technique used to measure a value.

The term “contacting” refers to mixing, adding, slurring, stirring a clear solution or a combination thereof. The term “contacting”, as used herein for the purpose of reaction, means wherein one or more reagent(s) are mixed or added with each other and/or solvent(s) in any sequences and further slurried as an insoluble solid mixture or stirred as a clear solution.

The term “about” is to be construed as modifying a term or value such that it is not an absolute. Such term will be defined by the circumstances. This includes, at the very least, the degree of expected experimental error, technique error and instrument error for a given technique used to measure a value.

The term “substantially free of impurities” means less than 0.2% of total impurity as measured by area percentage HPLC, preferably less than 0.1% of total impurities as measured by area percentage HPLC.

### ABBREVIATIONS

HPLC: High-performance liquid chromatography
¹H NMR: Proton nuclear magnetic resonance chromatography
¹³C NMR: Carbon-13 (¹³C) nuclear magnetic resonance chromatography
DPPCI: Diphenylphosphinic chloride
BMPCI: Bis-morpholinophosphorylchloride
CPTCI: Cyclopenta phosphoronic chloride
ACN: Acetonitrile
NMP: N-methyl pyrrolidone
DMCHDA: Trans-N, N′-Dimethylcyclohexane-1,2-diamine
KOBt: Potassium tertiary butoxide
NaOBt: Sodium tertiary butoxide
CuI: Copper Iodide
CuBr: Copper Bromide
EDC·HCl: 1-Ethyl-3-(3-dimethylaminopropyl)carbodiimide hydrochloride
DMAP: Dimethyl amino pyridine
TEA: Triethyl amine
DIPEA: N,N-Diisopropylethylamine
DBU: 1,8-Diazabicyclo(5.4.0)undec-7-ene
HOBt: Hydroxybenzotriazole
THF: Tetrahydrofuran
DMF: Dimethylformamide
MDC: Dichloromethane
TEAI: Tetraethylammonium iodide
Tris: Tromethamine
DCC: N,N′-Dicyclohexylcarbodiimide
Cbz: Carboxybenzyl
DMAC: Dimethylacetamide
KPH: Potassium hydrogen phthalate

The present invention provides novel Fostemsavir intermediates and processes for the preparation of novel Fostemsavir intermediates having additional advantages over the prior processes such as impurity control by washing out isomeric impurity at triazole insertion stage. The present invention also provides highly pure and stable Fostemsavir or pharmaceutically acceptable salts thereof and improves overall yield. The process for preparation of Fostemsavir Tromethamine using the novel intermediates of Fostemsavir has been depicted below as Scheme-3.

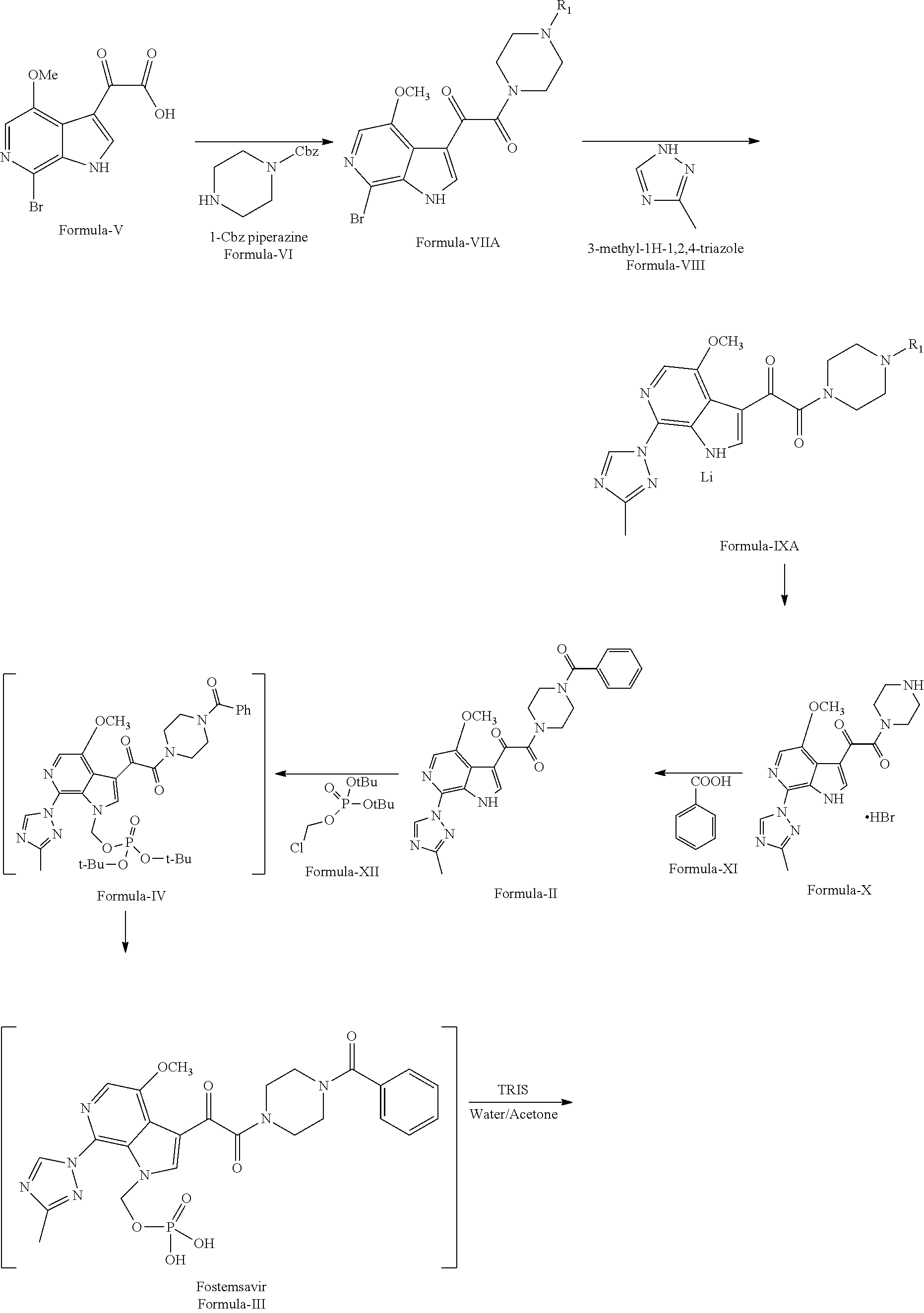

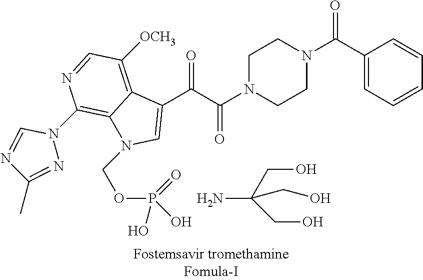

wherein R¹=

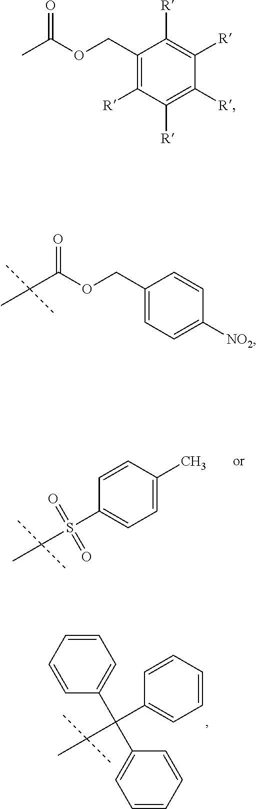

and
wherein
R′=H, F, Cl, Br, I, C₁-C₆ alkyl, Aryl, —NO₂, —OR″, —COOR″ or —NR″₂, and R″=H, C₁-C₆ alkyl or Aryl.

### Scheme-3: Process for the Preparation of Fostemsavir Tromethamine

In some embodiments, the invention relates to a novel Fostemsavir intermediate represented by Formula-VIIA:

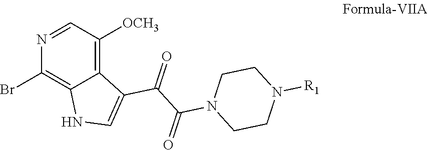

wherein R¹=

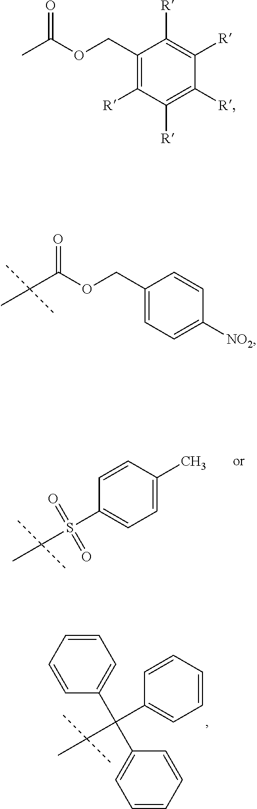

and
wherein
R′=H, F, Cl, Br, I, C₁-C₈ alkyl, Aryl, —NO₂, —OR″, —COOR″ or —NR″₂; and
R″=H, C₁-C₈ alkyl or Aryl.

In some of these embodiments, the Fostemsavir intermediate is represented by Formula-VII:

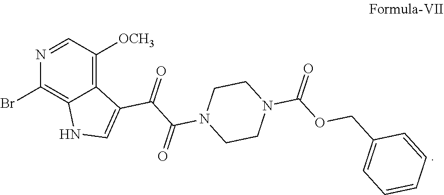 [SMILES: COc1cnc(Br)c2[nH]cc(C(=O)C(=O)N3CCN(C(=O)OCc4ccccc4)CC3)c12]

In some embodiments, the invention relates to a novel Fostemsavir intermediate represented by Formula-IXA:

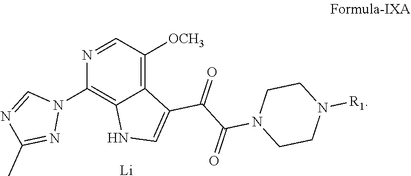

wherein R¹=

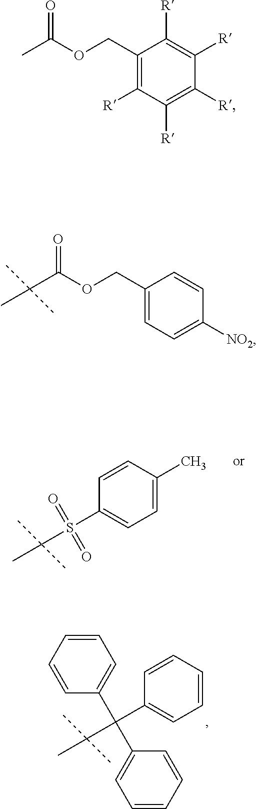

and
wherein
R′=H, F, Cl, Br, I, C₁-C₆ alkyl, Aryl, —NO₂, —OR″, —COOR″ or —NR″₂; and
R″=H, C₁-C₆ alkyl or Aryl.

In certain of these embodiments, the Fostemsavir intermediate is represented by Formula-IX:

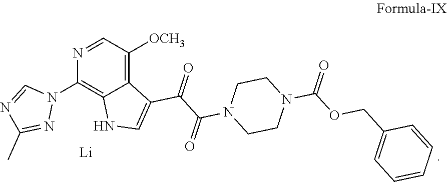

The novel process for the preparation of 1-(4-methoxy-7-(3-methyl-1H-1,2,4-triazol-1-yl)-1H-pyrrolo[2,3-c]pyridin-3-yl)-2-(piperazin-1-yl)ethane-1,2-dione of Formula-X may include the following steps:
(a) reacting 2-(7-bromo-4-methoxy-1H-pyrrolo[2,3-c]pyridin-3-yl)-2-oxoacetic acid of Formula-V with 1-Cbz piperazine of Formula-VI in presence of a coupling agent, a base and a solvent to obtain 1-(7-bromo-4-methoxy-1H-pyrrolo[2,3-c]pyridin-3-yl)-2-(4-substituted-piperazin-1-yl)ethane-1,2-dione of Formula-VIIA;
(b) reacting the compound of Formula-VIIA as obtained in step (a) with 3-Methyl-1H-1,2,4-triazole of Formula-VIII in presence of DMCHDA, a solvent, a base and a metal catalyst to obtain 1-(4-substituted-piperazin-1-yl)-2-(4-methoxy-7-(3-methyl-1H-1,2,4-triazol-1-yl)-1H-pyrrolo[2,3-c]pyridin-3-yl)ethane-1,2-dione, lithium salt of Formula-IXA; and
(c) reducing the compound of Formula-IXA in presence of a reagent and a solvent to obtain 1-(4-methoxy-7-(3-methyl-1H-1,2,4-triazol-1-yl)-1H-pyrrolo[2,3-c]pyridin-3-yl)-2-(piperazin-1-yl)ethane-1,2-dione of Formula-X.

The intermediate as obtained in step (c) may be further reacted to obtain Fostemsavir tromethamine of Formula-I as the final product, which comprises steps of:
(d) benzoylating the compound of Formula-X as obtained in step (c) with a coupling agent, a base and a solvent to obtain Temsavir of Formula-Il;
(e) treating the Temsavir of Formula-Il as obtained in step (d) with di-tert-butyl (chloromethyl) phosphate in presence of acetonitrile, potassium carbonate and tetraethylammonium iodide to obtain in-situ compound of Formula-IV;
(f) adding water and acetone to the in-situ compound of Formula-IV to obtain in-situ Fostemsavir of Formula-III; and
(g) converting the in-situ Fostemsavir of Formula-III to Fostemsavir tromethamine of Formula-I in presence of tromethamine.

In some embodiments, compounds other than 1-Cbz piperazine of Formula-VI may be used in step (a). For example, a substituted benzyloxycarbamoyl piperazine, such as p-nitrobenzyloxycarbamoyl (PNB) or p-methoxybenzyloxycabamoyl (PMB) piperazine, may be used in step (a). In additional embodiments, other protected derivatives, such as 1-tritylpiperazine or 1-toluenesulfonylpiperazine, may also be used in step (a). These alternative protecting groups can be removed in step (c) by appropriate methods to obtain 1-(4-methoxy-7-(3-methyl-1H-1,2,4-triazol-1-yl)-1H-pyrrolo[2,3-c]pyridin-3-yl)-2-(piperazin-1-yl)ethane-1,2-dione of Formula-X.

In some embodiments, the coupling agent used in step (a) is selected from DPPCI, BMPCI, CPTCI and a mixture thereof. In certain of these embodiments, the coupling agent is DPPCI.

In certain embodiments, the base used in step (a) is selected from 4-Methylmorpholine, TEA, DIPEA, DBU and a mixture thereof. In some of these embodiments, the base is 4-Methylmorpholine.

The solvent from step (a) may be selected from NMP, Acetonitrile, THF, DMF and a mixture thereof. In certain embodiments, the solvent is NMP.

In some embodiments, the solvent used in step (b) is be selected from DMAc, NMP, DMF and water, or a mixture thereof. In certain of these embodiments, the solvent is a mixture of DMAc and water.

The base used in step (b) may be selected from KOH, NaOH, KOBt, NaOBt or a mixture thereof. In certain preferred embodiments, the base is KOH.

The catalyst used in step (b) may be selected from CuI and CuBr. Preferably, the catalyst is CuI.

The reagent used in step (c) may be selected from hydrochloric acid, hydrobromic acid or mixture thereof. Preferably, the reagent is hydrobromic acid.

The solvent used in step (c) may be selected from acetic acid, trifluoro acetic acid, hydrofluoric acid or mixture thereof. In some preferred embodiments, the solvent is acetic acid.

The coupling agent used in step (d) may be selected from DPPCI, BMPCI, CPTCI or a mixture thereof. Preferably, the coupling agent may be DPPCI.

The base used in step (d) may be selected from 4-Methylmorpholine, TEA, DIPEA, DBU or a mixture thereof. In certain preferred embodiments, the base is 4-Methylmorpholine.

The solvent from step (d) may be selected from NMP, Acetonitrile, THF, DMF or a mixture thereof. Preferably, the solvent is NMP.

In the present invention, the inventors used two novel Cbz protected intermediate compounds. Compound of formula-IX is prepared from compound of formula-V via formation of compound of formula-VII. During the process, the inventors surprisingly observed that novel Cbz intermediates of the present invention play a key role in controlling the formation of triazole undesired isomer of compound of formula-XX. The comparison table is mentioned below:

Cbz route for preparation of compound of
formula-IX from compound of formula-V

| | Before Lithium salt | |
|---|---|---|
| Comparison Data | (Crude) | After Lithium salt |
| Yield (%) | 80% | 52% |
| Purity (%) | 90.2% | 99.6% |
| Undesired Isomer | 3.63% | 0.13% |
| (%) | | |

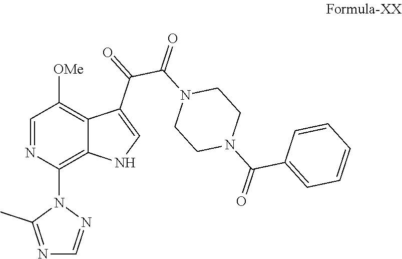 [SMILES: COc1cnc(-n2ncnc2C)c2[nH]cc(C(=O)C(=O)N3CCN(C(=O)c4ccccc4)CC3)c12]

The following nonlimiting examples illustrate the methods for preparing the Fostemsavir tromethamine and related compounds as discussed in the present invention.

### Examples

### Example 1: Preparation of Benzyl 4-(2-(7-bromo-4-methoxy-1H-pyrrolo[2,3-c]pyridin-3-yl)-2-oxoacetyl)piperazine-1-carboxylate

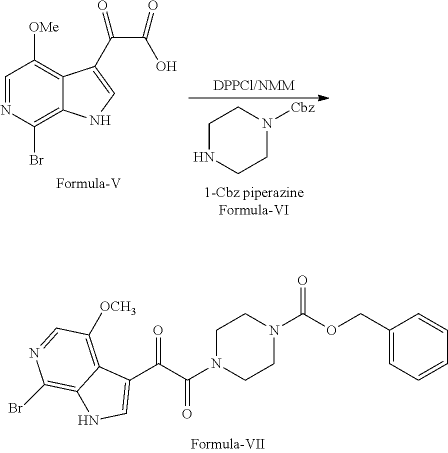 [SMILES: COc1cnc(Br)c2[nH]cc(C(=O)C(=O)O)c12 ; O=C(OCc1ccccc1)N1CCNCC1 ; COc1cnc(Br)c2[nH]cc(C(=O)C(=O)N3CCN(C(=O)OCc4ccccc4)CC3)c12]

To the stirred solution of 2-(7-bromo-4-methoxy-1H-pyrrolo[2,3-c]pyridin-3-yl)-2-oxoacetic acid (30 gm, 1.0 eq.), 1-Cbz piperazine (28.5 g, 1.1 eq.) and N-Methyl morpholine (50 gm, 2 eq.) in NMP (300 ml, 10V), a solution of Diphenylphosphinic chloride (28.5 g, 1.2 eq.) in NMP (30 ml, 1V) was added at 0-5° C. Then, the reaction mass was stirred at room temperature for about 8-10 hrs, then quenched with mixture of water and NMP (228 ml, 7.6V) and stirred for about 1 hr at 60° C. The reaction mass gradually cooled to room temperature and additional water was added. The precipitated solid was filtered off and washed in ACN (200 ml) to afford 38 gm dried pure benzyl 4-(2-(7-bromo-4-methoxy-1H-pyrrolo[2,3-c]pyridin-3-yl)-2-oxoacetyl)piperazine-1-carboxylate. Yield: 75%, ¹H NMR (400 MHZ, DMSO): δ 3.36 (s, 2H), 3.43 (s, 2H), 3.56 (s, 2H), 3.63 (s, 2H), δ 3.92 (s, 3H), 5.1 (s, 2H), 7.310-7.353 (m, 5H), δ 7.82 (s, 1H), 8.34 (s, 1H), ¹³C NMR (100 MHz, DMSO): 40.88, 45.48, 57.15, 66.95, 115.38, 117.44, 121.56, 124.21, 128.09, 128.35, 128.86, 133.74, 137.13, 138.83, 150.35, 154.87, 166.75, 185.92 Purity: 99.58%, m/z: 503.

### Example 2: Preparation of benzyl 4-(2-(4-methoxy-7-(3-methyl-1H-1,2,4-triazol-1-yl)-1H-pyrrolo[2,3-c]pyridin-3-yl)-2-oxoacetyl)piperazine-1-carboxylate, lithium salt

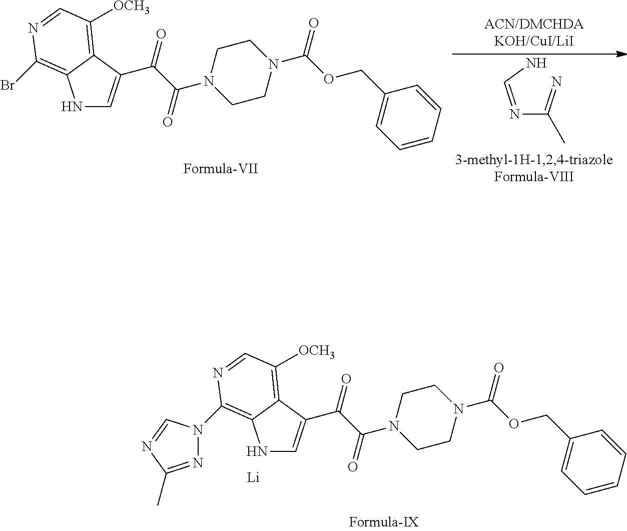

Potassium hydroxide (1.17 g) was dissolved in water (5.5 g) and DMAc (12.5 ml) at room temperature. To the stirred reaction mass, the solid obtained in example-1 (5 g), 3-Methyl-1H-1,2,4-triazole (1.046 g), DMCHDA (1.79 g) and catalytic CuI (0.03 g) were added. The reaction mass was maintained at 130° C. for 18 hrs. The reaction mass was quenched with mixture of purified water, KPH and ethyl acetate. Layers were separated and organic layers were treated with sodium sulphate and distilled off. After the distillation, water and MDC were added to the reaction mass and pH was adjusted 5 to 5.5 with acetic acid. Layers were separated and organic layers were treated with sodium sulphate and distilled off to afford 4.8 gm of crude product.

Lithium salt Preparation: Above 4.8 gm crude product was stirred in a mixture of ACN and water at 60° C. A solution of Lithium iodide (1.2 g) in ACN was slowly added at 60° C. and stirred for about 30 min. The reaction mass was gradually cooled to room temperature and precipitated solid was filtered off and washed ACN (200 ml) to afford 2.6 g dried pure benzyl 4-(2-(4-methoxy-7-(3-methyl-1H-1,2,4-triazol-1-yl)-1H-pyrrolo[2,3-c]pyridin-3-yl)-2-oxoacetyl)piperazine-1-carboxylate, lithium salt. Yield: 51.5%, ¹H NMR (400 MHZ, DMSO): δ 2.48 (s, 3H), δ 3.39 (s, 2H), 3.44 (s, 2H), 3.56 (s, 2H), 3.64 (s, 2H), δ 3.96 (s, 3H), 5.11 (s, 2H), 7.317 (s, 5H), δ 7.83 (s, 1H), 8.24 (s, 1H), δ 9.22 (s, 1H), δ 12.37 (b, 1H), ¹³C NMR (100 MHz, DMSO): 14.33, 43.87, 45.49, 57.25, 66.96, 114.62, 121.33, 123.26, 124.39, 128.13, 128.38, 128.89, 130.14, 137.15, 139.36, 142.66, 149.64, 154.87, 157.48, 161.79, 166.83, 185.96. Purity: 99.56%, isomer: 0.16%, m/z: 504

### Example 3: Preparation of 1-(4-methoxy-7-(3-methyl-1H-1,2,4-triazol-1-yl)-1H-pyrrolo[2,3-c]pyridin-3-yl)-2-(piperazin-1-yl)ethane-1,2-dione

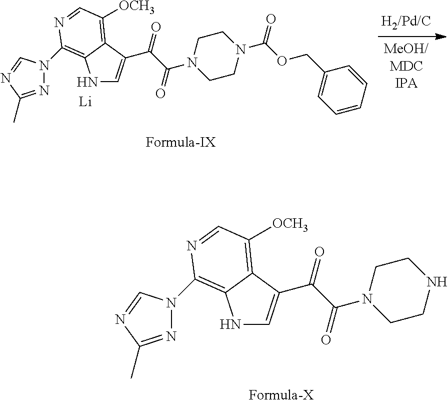

The solid obtained in example-2 (4.0 g) was dissolved in mixture of methanol (40 ml) and MDC (40 ml) and treated with activated carbon at room temperature. A solution of the reaction mass and ˜50% wet 10% Pd/C were loaded in the autoclave under nitrogen atmosphere. Hydrogen gas pressure was applied to the reaction mass and 4-5 Kg/cm² pressure was maintained until reaction completed. After completion of reaction, Pd/C was filtered and filtrate was distilled out under vacuum. IPA (10 ml) was added to the distilled reaction mass and stirred for about 30 min at room temperature. Precipitated solid was filtered off and washed with IPA (4 ml) to afford 2.6 g dried pure 1-(4-methoxy-7-(3-methyl-1H-1,2,4-triazol-1-yl)-1H-pyrrolo[2,3-c]pyridin-3-yl)-2-(piperazin-1-yl)ethane-1,2-dione. Yield: 92%, m/z: 370.

### Example 4: Preparation of 1-(4-methoxy-7-(3-methyl-1H-1,2,4-triazol-1-yl)-1H-pyrrolo[2,3-c]pyridin-3-yl)-2-(piperazin-1-yl)ethane-1,2-dione

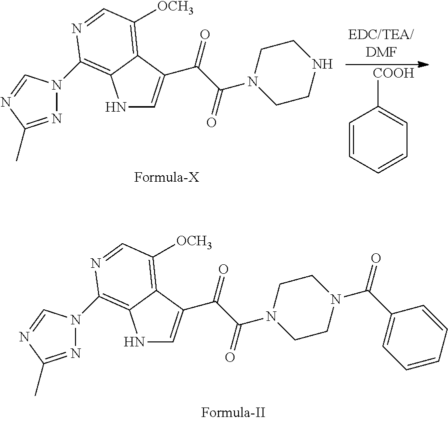 [SMILES: COc1cnc(-n2cnc(C)n2)c2[nH]cc(C(=O)C(=O)N3CCNCC3)c12 ; O=C(O)c1ccccc1 ; COc1cnc(-n2cnc(C)n2)c2[nH]cc(C(=O)C(=O)N3CCN(C(=O)c4ccccc4)CC3)c12]

To a solution of amine obtained in Example 3 (1.0 g, 1 eq.) and Benzoic acid (0.33 gm, 1 eq.) in DMF (3.4 ml, 17V) was added Triethyl amine (2.6 g, 10 eq.) followed by 1-[w3-(dimethylamino)propyl]-3-ethylcarbodiimide hydrochloride (EDC·HCl) (1.55 g, 3 eq.) under nitrogen and the reaction mixture was stirred at room temperature for about 16 hrs. After completion of the reaction, DMF and TEA were distilled out under vacuum and water was added to the distilled reaction mass. Precipitated solid was filtered off and washed with water (4 ml) to obtain 0.8 g dried pure 1-(4-methoxy-7-(3-methyl-1H-1,2,4-triazol-1-yl)-1H-pyrrolo[2,3-c]pyridin-3-yl)-2-(piperazin-1-yl)ethane-1,2-dione. Yield: 62.5%, m/z: 474.

### Example 5: Preparation of 1-(4-methoxy-7-(3-methyl-1H-1,2,4-triazol-1-yl)-1H-pyrrolo[2,3-c]pyridin-3-yl)-2-(piperazin-1-yl)ethane-1,2-dione hydrobromide

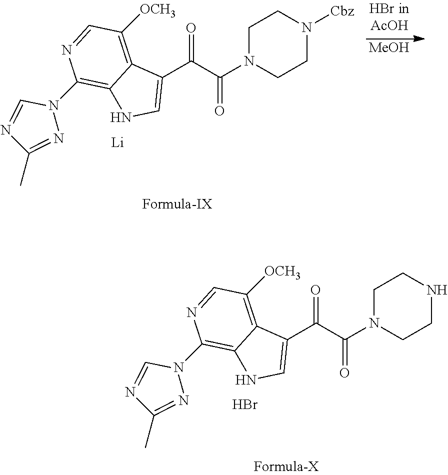

To a cooled solution of Acetic acid (60 ml) and 30% Hydrobromic acid in acetic acid (60 ml), the solid obtained as per example-2 (20.0 g) was added portion wise at about 18-20° C. The reaction mass was maintained at 25°-30° C. for about 6-8 hrs. Methanol (300 ml) was added to the reaction mass and stirred for 30 min at room temperature. Precipitated solid was filtered off and washed with methanol (40 ml) to afford 17 g dried pure 1-(4-methoxy-7-(3-methyl-1H-1,2,4-triazol-1-yl)-1H-pyrrolo[2,3-c]pyridin-3-yl)-2-(piperazin-1-yl)ethane-1,2-dione hydrobromide. Yield: 96%, Purity: 99.07%, ¹H NMR (400 MHZ, DMSO): δ 2.48 (s, 3H), 3.16 (s, 2H), 3.28 (s, 2H), 3.65 (s, 2H), 3.87 (s, 2H), 3.98 (s, 3H), 7.82 (s, 1H), 8.29 (s, 1H), 9.27 (s, 2H), δ 9.61 (s, 1H), 12.45 (s, 1H), ¹³C NMR (100 MHz, DMSO): 13.64, 37.93, 42.44, 42.80, 43.08, 57.56, 114.36, 121.37, 123.16, 124.17, 129.43, 139.46, 149.84, 166.78, 185.46. m/z: 370.

### Example 6: Preparation of 1-(4-methoxy-7-(3-methyl-1H-1,2,4-triazol-1-yl)-1H-pyrrolo[2,3-c]pyridin-3-yl)-2-(piperazin-1-yl)ethane-1,2-dione

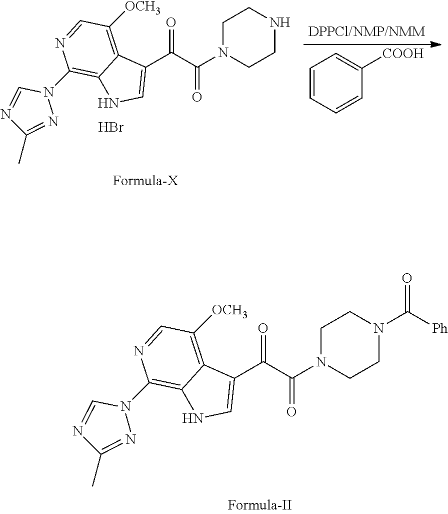

To the stirred solution of 1-(4-methoxy-7-(3-methyl-1H-1,2,4-triazol-1-yl)-1H-pyrrolo[2,3-c]pyridin-3-yl)-2-(piperazin-1-yl)ethane-1,2-dione hydrobromide (20 g, 1.0 eq.), Benzoic acid (6 g, 1.1 eq.) and N-Methyl morpholine (22.45 g, 5 eq.) in NMP (280 ml, 14V), a solution of Diphenylphosphinic chloride (13.65 g, 1.3 eq.) in NMP (20 ml, 1V) was slowly added at 0-5° C. The reaction mass stirred at room temperature for about 8-10 hrs, then quenched with water (40 ml, 2V) and stirred for about 1 hr at 60° C. with ethyl acetate (200 ml, 10V). The reaction mass gradually cooled to room temperature and stirred for about 30 min. Precipitated solid was filtered off and washed ethyl acetate (40 ml) to afford 17 g dried pure 1-(4-methoxy-7-(3-methyl-1H-1,2,4-triazol-1-yl)-1H-pyrrolo[2,3-c]pyridin-3-yl)-2-(piperazin-1-yl)ethane-1,2-dione. Yield: 81%, Purity: 99.13%, ¹H NMR (400 MHZ, DMSO): δ 2.50 (s, 3H), δ 3.44 (b, 4H), 3.67 (b, 4H), δ 3.93 (s, 3H), 7.46 (s, 5H), δ 7.89 (s, 1H), 8.25 (s, 1H), δ 9.25 (s, 1H), δ 12.42 (s, 1H), ¹³C NMR (100 MHZ, DMSO): 14.36, 57.34, 114.67, 121.48, 124.12, 127.57, 128.93, 130.20, 139.10, 142.70, 149.71, 161.87, 166.74, 169.48. m/z: 474.

Fostemsavir tromethamine of formula-I may be produced from Temsavir of formula-Il by methods well known to those skill in the art. Examples of such methods include those described in U.S. Pat. No. 7,745,625.

It should be noted that the invention in its broader aspects is not limited to the specific details, representative compositions, methods, and processes, and illustrative examples described in connection with the preferred embodiments and preferred methods. Modifications and equivalents will be apparent to practitioners skilled in this art and are encompassed within the spirit and scope of the appended claims.

What is claimed is:

1. A compound of formula-VIIA: 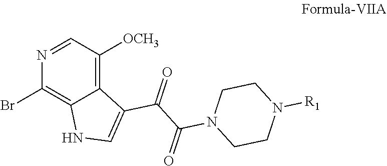
wherein R₁ is selected from 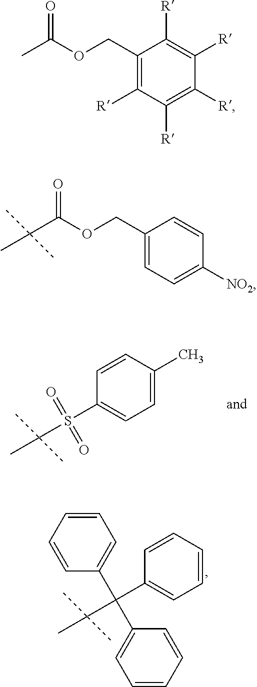
and
wherein:
R′ is selected from H, F, Cl, Br, I, C₁-C₈ alkyl, Aryl, —NO₂, —OR″, —COOR″ and —NR″₂; and
R″ is selected from H, C₁-C₆ alkyl and Aryl.

2. The compound according to claim 1, wherein Riis 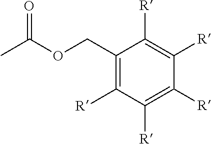
and R′ is H.

3. A compound of formula-IXA: 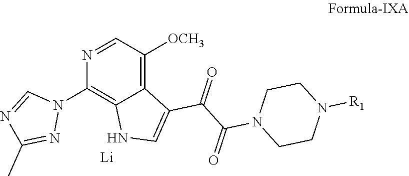
wherein R₁ is selected from 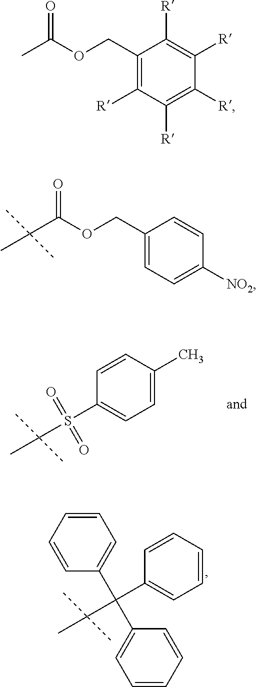
and
wherein:
R′ is selected from H, F, Cl, Br, I, C₁-C₈ alkyl, Aryl, —NO₂, —OR″, —COOR″ and —NR″₂; and
R″ is selected from H, C₁-C₆ alkyl and Aryl.

4. The compound according to claim 3, wherein R₁ is 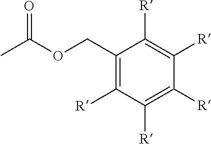
and R′ is H.

5. A process for the preparation of 1-(4-methoxy-7-(3-methyl-1H-1,2,4-triazol-1-yl)-1H-pyrrolo[2,3-c]pyridin-3-yl)-2-(piperazin-1-yl)ethane-1,2-dione of Formula-X: 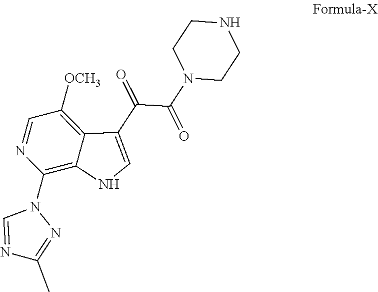 [SMILES: COc1cnc(-n2cnc(C)n2)c2[nH]cc(C(=O)C(=O)N3CCNCC3)c12]
comprising steps of:
a) reacting 2-(7-bromo-4-methoxy-1H-pyrrolo[2,3-c]pyridin-3-yl)-2-oxoacetic acid of Formula-V 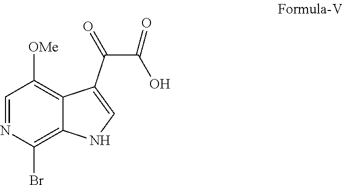 [SMILES: COc1cnc(Br)c2[nH]cc(C(=O)C(=O)O)c12]
with 1-Cbz piperazine of Formula-VI 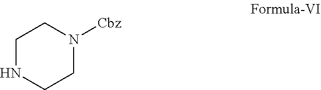 [SMILES: O=C(OCc1ccccc1)N1CCNCC1]
in presence of a coupling agent, a base and a solvent to obtain a compound of Formula-VIIA 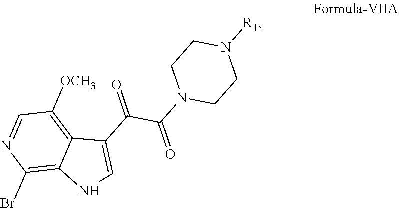
wherein R₁ is 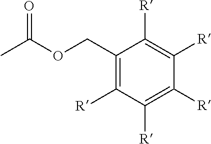
and R′ is H;
b) reacting the compound of Formula-VIIA with 3-methyl-1H-1,2,4-triazole of Formula-VIII 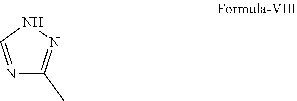
in presence of trans-N,N′-dimethylcyclohexane-1,2-diamine, a solvent, a base and a metal catalyst to obtain benzyl compound of Formula-IXA 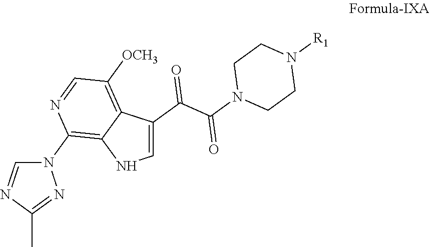
wherein R₁ is 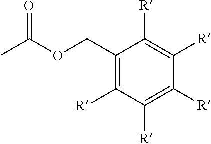
and R′ is H;
c) reducing the compound of Formula-IXA in presence of a reagent and a solvent to obtain compound of Formula-X.

6. The process according to claim 5, further comprising the step of:
d) benzoylating the compound of Formula-X with a coupling agent, a base and a solvent to obtain temsavir of Formula-Il.

7. The process according to claim 5, wherein the coupling agent used in step (a) and step (d) is selected from diphenylphosphinic chloride, bis-morpholinophosphorylchloride, Cyclopenta phosphoronic chloride and a mixture thereof.

8. The process according to claim 7, wherein the coupling agent used in step (a) and step (d) is diphenylphosphinic chloride.

9. The process according to claim 5, wherein the base used in step (a) and step (d) is selected from 4-Methylmorpholine, triethyl amine, N,N-diisopropylethylamine, 1,8-diazabicyclo[5.4.0]undec-7-ene and a mixture thereof.

10. The process according to claim 9, wherein the base used in step (a) and step (d) is 4-Methylmorpholine.

11. The process according to claim 5, wherein the solvent used in step (a) and step (d) is selected from N-methyl pyrrolidone, acetonitrile, tetrahydrofuran, dimethylformamide and a mixture thereof.

12. The process according to claim 5, wherein the solvent used in step (b) is selected from dimethylacetamide, N-methyl pyrrolidone, dimethylformamide, water, and a mixture thereof.

13. The process according to claim 5, wherein the base used in step (b) is selected from KOH, NaOH, potassium tertiary butoxide, and sodium tertiary butoxide.

14. The process according to claim 13, wherein the base used in step (b) is KOH.

15. The process according to claim 5, wherein the catalyst used in step (b) is selected from copper iodide and copper bromide.

16. The process according to claim 15, wherein the catalyst used in step (b) is copper iodide.

17. The process according to claim 5, wherein the solvent used in step (c) is selected from acetic acid, trifluoro acetic acid, hydrofluoric acid, and a mixture thereof.

18. The process according to claim 17, wherein the solvent used in step (c) is acetic acid.

19. The process according to claim 5, wherein the reagent used in step (c) is selected from hydrochloric acid, hydrobromic acid, and a mixture thereof.

20. The process according to claim 19, wherein the reagent used in step (c) is hydrobromic acid.
